(19) United States
(12) Patent Application Publication
(10) Pub. No.: US 2023/0354909 A1
(43) Pub. Date: Nov. 9, 2023

(54) AEROSOL PROVISION DEVICE AND AEROSOL PROVISION SYSTEM

(71) Applicant: Nicoventures Trading Limited, LONDON (GB)

(72) Inventors: Nicholas ROSSER, London (GB); Connor BRUTON, London (GB); Charanjit Nandra, London (GB); David RUSHFORTH, London (GB); Darryl BAKER, London (GB); Robert KERSEY, London (GB); Mark CROSIER, London (GB)

(21) Appl. No.: 18/004,929

(22) PCT Filed: Jul. 6, 2021
(86) PCT No.: PCT/GB2021/051715

§ 371 Date: Jan. 10, 2023

(30) Foreign Application Priority Data
Jul. 10, 2020 (GB) 2010619.1

(51) Int. Cl. A24F 40/51; A24F 40/57; A24F 40/53; G01P 13/00

(52) U.S. Cl. CPC A24F 40/51; A24F 40/57; A24F 40/53; G01P 13/0073

(57) ABSTRACT

An aerosol provision device includes a microphone configured to detect a flow of air in the device and to output corresponding airflow detection signals to control circuitry. The control circuitry is configured to determine a duration of an inhalation by a user of the aerosol delivery device based on the airflow detection signals received from the microphone.

### PRIORITY CLAIM

The present application is a National Phase entry of PCT Application No. PCT/GB2021/051715, filed Jul. 6, 2021, which claims priority from GB Application No. 2010619.1, filed Jul. 10, 2020, each of which hereby fully incorporated herein by reference.

### TECHNICAL FIELD

The present disclosure relates to an aerosol provision device and an aerosol provision system.

### BACKGROUND

Electronic aerosol provision systems such as electronic cigarettes (e-cigarettes) generally contain an aerosol-generating material, such as a reservoir of a source liquid containing a formulation, typically including nicotine, or a solid material such as a tobacco-based product, from which an aerosol is generated for inhalation by a user, for example through heat vaporization. Thus, an aerosol provision system will typically comprise an aerosol generator, e.g. a heating element, arranged to aerosolize a portion of aerosol-generating material to generate an aerosol in an aerosol generation region of an air channel through the aerosol provision system. As a user inhales on the device and electrical power is supplied to the aerosol generator, air is drawn into the device through one or more inlet holes and along the air channel to the aerosol generation region, where the air mixes with the vaporized aerosol generator and forms a condensation aerosol. The air drawn through the aerosol generation region continues along the air channel to a mouthpiece, carrying some of the aerosol with it, and out through the mouthpiece for inhalation by the user.

It is common for aerosol provision systems to comprise a modular assembly, often having two main functional parts, namely an aerosol provision device and disposable/replaceable consumable part. Typically the consumable will comprise the consumable aerosol-generating material and the aerosol generator (heating element), while the aerosol provision device part will comprise longer-life items, such as a rechargeable battery, device control circuitry and user interface features. The aerosol provision device may also be referred to as a reusable part or battery section and the consumable may also be referred to as a disposable part, cartridge or cartomizer.

The aerosol provision device and consumable are mechanically coupled together at an interface for use, for example using a screw thread, bayonet, latched or friction fit fixing. When the aerosol-generating material in a consumable has been exhausted, or the user wishes to switch to a different consumable having a different aerosol-generating material, the consumable may be removed from the aerosol provision device and a replacement consumable may be attached to the device in its place.

A potential drawback for aerosol provision systems is that the device can activate when it is not intended to be used by the user. For example, the user may inadvertently turn on the device by providing an input, such as a button press, when the user is not intending to use the device for aerosol generation or inhalation. In this case, electrical power is supplied to the aerosol generator without air flow through the air channel. This reduces the charge in the battery, thereby requiring the battery to be charged more frequently, and can require the consumable to be replaced more regularly due to overheating or drying out of the aerosol generator or premature depletion of the aerosol-generating material.

Various approaches are described herein which seek to help address or mitigate some of the issues discussed above.

### SUMMARY

In accordance with some embodiments described herein, there is provided an aerosol provision device comprising a microphone configured to detect a flow of air in the aerosol provision device and to output corresponding airflow detection signals to control circuitry, and control circuitry configured to determine a duration of an inhalation by a user of the aerosol delivery device based on the airflow detection signals received from the microphone.

The control circuitry may be configured to determine a time between inhalations based on the airflow detection signals.

The microphone may be configured to detect the flow of air in the aerosol provision device based on a noise level exceeding a detection threshold, and the control circuitry may be configured to determined the duration of the inhalation by activating an inhalation timer in response to receiving the airflow detection signals from the microphone.

The microphone may be configured to stop outputting airflow detection signals when the noise level does not exceed the detection threshold, and the control circuitry may be configured to determine a time between inhalations by activating an interval timer when the microphone stops outputting the airflow detection signals.

The control circuitry may be configured to control an aerosol generator based on the airflow detection signals, such as activating the aerosol generator in response to the airflow detection signals exceeding an activation threshold.

The control circuitry may be configured to detect a first phase of the inhalation and a second phase of the inhalation based on the airflow detection signals, such as determining the duration of the first phase of the inhalation and duration of the second phase of the inhalation based on the airflow detection signals. The first phase of the inhalation may occur until the airflow detection signals are above a phase threshold and the second phase of the inhalation may occur after the airflow detection signals are above the phase threshold.

The control circuitry may be configured to determine an amount of an ingredient delivered from an aerosol-generating material to the user during the inhalation based on the airflow detection signals and the control circuitry may be configured to determine an amount of an ingredient delivered from an aerosol-generating material to the user during the inhalation based on the duration of the first phase of the inhalation and duration of the second phase of the inhalation. The ingredient may be nicotine.

The control circuitry is configured to determine default inhalation behavior for the user based on the airflow detection signals received for a plurality of inhalations, and the control circuitry may be configured to alter the activation threshold based on the determined default inhalation behavior and/or the phase threshold based on the determined default inhalation behavior.

In accordance with some embodiments described herein, there is provided an aerosol provision system comprising the aerosol provision device described herein.

In accordance with some embodiments described herein, there is provided an aerosol provision system comprising a microphone configured to detect a flow of air in the aerosol provision device and to output corresponding airflow detection signals to control circuitry, and control circuitry configured to determine a duration of an inhalation by a user of the aerosol provision device based on the airflow detection signals received from the microphone.

In accordance with some embodiments described herein, there is provided a method of determining a duration of an inhalation by a user of an aerosol provision device comprising receiving airflow detection signals from a microphone configured to detect a flow of air in the aerosol provision device and determining a duration of an inhalation by a user of the aerosol provision device based on the airflow detection signals received from the microphone. There is also provided a computer readable storage medium comprising instructions which, when executed by a processor, performs the above method.

These aspects and other aspects will be apparent from the following detailed description. In this regard, particular sections of the description are not to be read in isolation from other sections.

### BRIEF DESCRIPTION OF DRAWINGS

Embodiments of the disclosure will now be described, by way of example only, with reference to accompanying drawings, in which:

FIG. 1 is a schematic diagram of an aerosol provision system.

FIGS. 2A-C are schematic diagrams of example aerosol provision devices for use in the aerosol provision system illustrated in FIG. 1.

FIG. 3A is a graph of noise detected by a microphone against time.

FIGS. 3B and 3C are graphs of airflow detection signals output by a microphone against time.

FIGS. 4A to 4C are graphs of airflow detection signals output by a microphone against time.

FIG. 5 is a flow chart of a method of determining a duration of an inhalation by a user of an aerosol provision device.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of articles and systems discussed herein which are not described in detail may be implemented in accordance with any conventional techniques for implementing such aspects and features.

The present disclosure relates to aerosol provision systems, which may also be referred to as aerosol provision systems, such as e-cigarettes. Throughout the following description the term “e-cigarette” or “electronic cigarette” may sometimes be used, but it will be appreciated this term may be used interchangeably with aerosol provision system and electronic aerosol provision system.

As noted above, aerosol provision systems (e-cigarettes) often comprise a modular assembly including both a reusable part (aerosol provision device) and a replaceable (disposable) cartridge part, referred to as a consumable. Systems conforming to this type of two-part modular configuration may generally be referred to as two-part systems or devices. It is also common for electronic cigarettes to have a generally elongate shape. For the sake of providing a concrete example, certain embodiments of the disclosure described herein comprise this kind of generally elongate two-part system employing disposable cartridges. However, it will be appreciated the underlying principles described herein may equally be adopted for other electronic cigarette configurations, for example modular systems comprising more than two parts, as devices conforming to other overall shapes, for example based on so-called box-mod high performance devices that typically have a more boxy shape.

As described above, the present disclosure relates to (but it not limited to) aerosol provision devices and corresponding aerosol provision systems, such as e-cigarettes and electronic cigarettes.

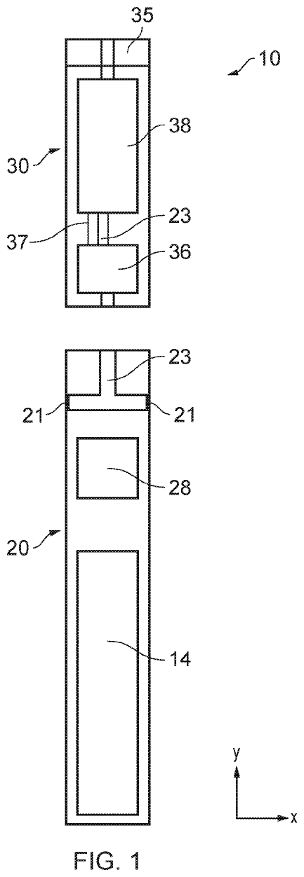

FIG. 1 is a highly schematic diagram (not to scale) of an example aerosol provision system 10, such as an e-cigarette, to which embodiments are applicable. The aerosol provision system has a generally cylindrical shape, extending along a longitudinal or y axis as indicated by the axes (although aspects of the disclosure are applicable to e-cigarettes configured in other shapes and arrangements), and comprises two main components, namely an aerosol provision device 20 and a consumable 30.

The consumable 30 is an article comprising or consisting of aerosol-generating material 38, part or all of which is intended to be consumed during use by a user. A consumable 30 may comprise one or more other components, such as an aerosol-generating material storage area, an aerosol-generating material transfer component 37, an aerosol generation area, a housing, a wrapper, a mouthpiece 35, a filter and/or an aerosol-modifying agent.

A consumable 30 may also comprise an aerosol generator 36, such as a heating element, that emits heat to cause the aerosol-generating material 38 to generate aerosol in use. The aerosol generator 36 may, for example, comprise combustible material, a material heatable by electrical conduction, or a susceptor. It should be noted that it is possible for the aerosol generator 36 to be part of the aerosol provision device 20 and the consumable 30 then may comprise the aerosol-generating material storage area for the aerosol-generating material 38 such that, when the consumable 30 is coupled with the aerosol provision device 20, the aerosol-generating material 38 can be transferred to the aerosol generator 36.

The aerosol-generating material 38 is a material that is capable of generating aerosol, for example when heated, irradiated or energized in any other way. The aerosol-generating material 38 may, for example, be in the form of a solid, liquid or gel which may or may not contain an active substance and/or flavorants. In some embodiments, the aerosol-generating material 38 may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material 38 comprises one or more ingredients, such as one or more active substances and/or flavorants, one or more aerosol-former materials, and optionally one or more other functional materials such as pH regulators, coloring agents, preservatives, binders, fillers, stabilizers, and/or antioxidants.

The active substance as used herein may be a physiologically active material, which is a material intended to achieve or enhance a physiological response. The active substance may for example be selected from nutraceuticals, nootropics, psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

In some embodiments, the active substance comprises nicotine. In some embodiments, the active substance comprises caffeine, melatonin or vitamin B12.

The aerosol provision device 20 includes a power source 14, such as a battery, configured to supply electrical power to the aerosol generator 36. The power source 14 in this example is rechargeable and may be of a conventional type, for example of the kind normally used in electronic cigarettes and other applications requiring provision of relatively high currents over relatively short periods. The battery 14 may be recharged through the charging port (not illustrated), which may, for example, comprise a USB connector.

The aerosol provision device 20 includes control circuitry 28 configured to control the operation of the aerosol provision system 10 and provide conventional operating functions in line with the established techniques for controlling aerosol provision systems such as electronic cigarettes. The control circuitry (processor circuitry) 28 may be considered to logically comprise various sub-units/circuitry elements associated with different aspects of the electronic cigarette's operation. For example, depending on the functionality provided in different implementations, the control circuitry 28 may comprises power source control circuitry for controlling the supply of electrical power from the power source 14 to the aerosol generator 36, user programming circuitry for establishing configuration settings (e.g. user-defined power settings) in response to user input, as well as other functional units/circuitry associated functionality in accordance with the principles described herein and conventional operating aspects of electronic cigarettes. It will be appreciated the functionality of the control circuitry 28 can be provided in various different ways, for example using one or more suitably programmed programmable computer(s) and/or one or more suitably configured application-specific integrated circuit(s)/circuitry/chip(s)/chipset(s) configured to provide the desired functionality.

The aerosol provision device 20 includes one or more air inlets 21. In use, as a user inhales on the mouthpiece 35, air is drawn into the aerosol provision device 20 through the air inlets 21 and along an air channel 23 to the aerosol generator 36, where the air mixes with the vaporized aerosol-generating material 38 and forms a condensation aerosol. The air drawn through the aerosol generator 36 continues along the air channel 23 to a mouthpiece 35, carrying some of the aerosol with it, and out through the mouthpiece 35 for inhalation by the user.

By way of a concrete example, the consumable 30 comprises a housing (formed, e.g., from a plastics material), a reservoir formed within the housing for containing the aerosol-generating material 38 (which in this example may be a liquid which may or may not contain nicotine), an aerosol-generating material transfer component 37 (which in this example is a wick formed of e.g., glass or cotton fibers, or a ceramic material configured to transport the liquid from the reservoir using capillary action), an aerosol generating area, and a mouthpiece 35. Although not shown, a filter and/or aerosol modifying agent (such as a flavor imparting material) may be located in, or in proximity to, the mouthpiece 35. The consumable of this example comprises a heater element formed from an electrically resistive material (such as NiCr8020) spirally wrapped around the aerosol-generating material transfer component 37, and located in the air channel 23. The area around the heating element and wick combination is the aerosol generating area of the consumable 30. The consumable comprises suitable electrical contacts for coupling to electrical contacts provided on the aerosol provision device 20, such that electrical power may be supplied directly to the heater element.

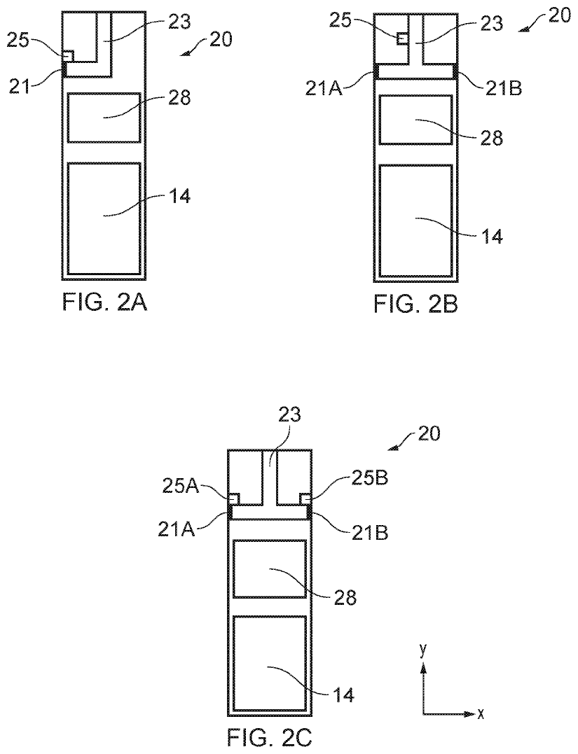

FIGS. 2A-C are schematic diagrams of example aerosol provision devices 20 for use in the aerosol provision system 10 illustrated in FIG. 1, where the same reference signs have been used for like elements between the aerosol provision devices 20 illustrated in FIG. 1 and the aerosol provision device 20 illustrated in FIGS. 2A-C. As per the aerosol provision device 20 illustrated in FIG. 1, the aerosol provision devices 20 illustrated in FIGS. 2A-C include a power source 14, control circuitry 28 and one or more air inlets 21. For example, the aerosol provision device 20 illustrated in FIG. 2A has one air inlet 21, whilst the aerosol provision device 20 illustrated in FIGS. 2B and 2C has two air inlets 21A, 21B. It will be appreciated that aerosol provision device 20 may have more than two air inlets 21, and that the number of air inlets 21 is not essential to the aspects of the disclosure described herein.

In the examples illustrated in FIGS. 2A-C, the aerosol provision device 20 includes a microphone 25, such as a MicroElectrical-Mechanical System (MEMS) microphone. The microphone 25 is configured to detect a flow of air in the aerosol provision device 20. In other words, the microphone 25 is configured to detect a noise associated with the flow of air in the aerosol provision device 20. For example, the microphone 25 may be configured to detect a flow of air into one or more of the air inlets 21. As illustrated in FIG. 2A, the microphone 25 may be located proximate to the air inlet 21 in order to detect a noise associated with the flow of air into the aerosol provision device 20 through the air inlet 21. Alternatively, or in addition, the microphone 25 may be configured to detect a flow of air in the air channel 23 through the aerosol provision device 20. As illustrated in FIG. 2B, the microphone may be located proximate to a portion of the air channel 23 in order to detect a noise associated with the flow of air through the aerosol provision device 20. Although not illustrated in FIG. 2, the microphone may be located in the consumable 30 in order to detect a noise associated with the flow of air through the consumable 30, for example the portion of the air channel 23 located in consumable 30 such as proximate to the aerosol generator 36 or the mouthpiece 35. In other words, the microphone 25 is configured to detect a flow noise of the flow of air caused by turbulent boundary-layer flow resulting from air passing into one or more of the air inlets 21 or air travelling through the air channel 23 or in/around the aerosol generator 36. Such microphones provide an accurate means of detecting flow through the aerosol provision device 10 compared to conventional methods. For example, the lag between the creation of the noise associated with the flow of air in the aerosol provision device 20 and the detection of the noise by the microphone 25 is relatively low compared to other methods, such as detecting a cooling of a heated wire due to the flow of air around the wire.

As illustrated in FIG. 2C, in some examples there is more than one microphone 25. In the example illustrated in FIG. 2C, there is a microphone 25A located proximate to a first air inlet 21A and a microphone 25B located proximate to the second air inlet 21B. Although not illustrated, there may be additional microphones within the aerosol provision device 20 and/or the consumable 30, such as proximate to a portion of the air channel 23, the aerosol generator 36 and/or the mouthpiece 35 as described above. Accordingly, each microphone is configured to detect a flow of air in the aerosol provision device 20. Additionally, a microphone may be located away from the air channel 23, for example at the end of the aerosol provision device distal to the consumable 30 in order to detect the ambient noise level around the aerosol provision device 20 for comparison with the noise level detected by the microphone proximate to the air inlet 21 or air channel 23, thereby improving the accuracy of the air flow detection by the microphones.

In some embodiments, there are one or more other types of sensor also configured to detect the flow of air in the aerosol provision device 20. For example, a flow sensor may also be used to detect the flow of air in the aerosol provision device 20. Alternatively or in addition, a temperature sensor, such as a heated negative temperature coefficient (NTC) resistive sensor could be used to detect air flow through the aerosol provision device 20. In this example, an ambient NTC sensor can be used in the aerosol provision device 20 to calibrate the heated NTC sensor.

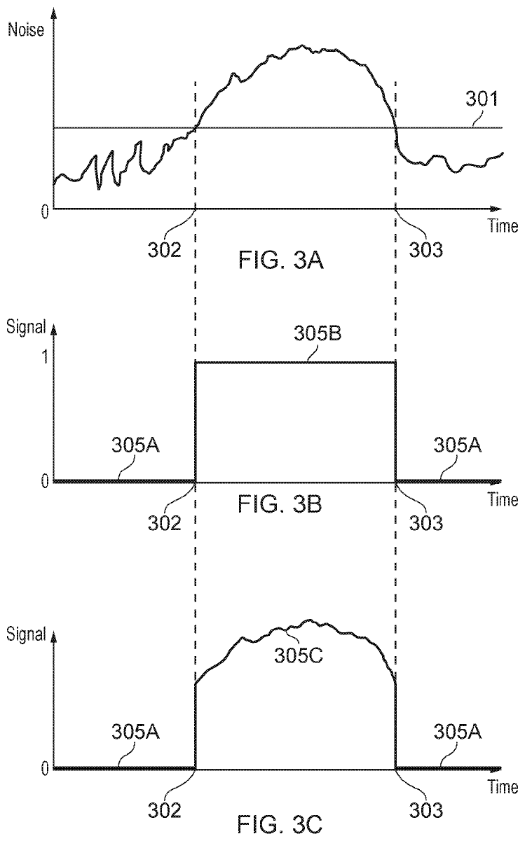

In some embodiments, the microphone 25 is configured to detect the flow of air in the aerosol provision device 20. The microphone 25 may include a processor or similar element to determine when there is a flow of air in the aerosol provision device 20 based on a noise level exceeding a detection threshold. The microphone 25 may output a signal (e.g., to the control circuitry 28) indicating the presence of a flow of air in the aerosol provision device 20. In other words, the microphone 25 is configured to detect a noise, and determine that the noise corresponds to the flow of air in the aerosol provision device 20 when the noise level exceeds a detection threshold. This is illustrated in FIG. 3A, which is a graph of noise detected by a microphone 25 against time. The horizontal line 301 corresponds to the detection threshold. The portion of the graph where the noise is above the detection threshold 301, corresponding to the portion between time points 302 and 303, corresponds to the portion of the noised determined by the microphone 25 to correspond to the flow of air in the aerosol provision device 20. In alternative implementations, the microphone may output a raw noise signal and the control circuitry 28 may be configured to determine whether the raw noise signal exceeds the detection threshold.

The detection threshold may be based on a sensitivity of the microphone 25, for example a minimum noise level, such as in decibels, that the microphone 25 is capable of detecting. For example, the detection threshold could be equal to the minimum noise level the microphone 25 is capable of detecting, for example −45, −40 or −30 dB, or the detection threshold could be a prescribed amount above the minimum noise level the microphone 25 is capable of detecting, such as 1, 5, 10 or 25 dB. Alternatively, the detection threshold may be based on the flow noise associated with a particular air speed/mass flow of the flow of air through the air channel 23 or into the air inlets 21. For example, the detection threshold may be the flow noise associated with an air speed of 2.0, 3.0 or 4.0 m/s through the air channel 23, or the detection threshold may be the flow noise associated with an inhalation or puff by the user, such as 50 ml over a duration of 3 seconds, or 80 ml over a duration of 4 seconds or 100 ml over a duration of 5 seconds.

In response to detecting a flow of air in the aerosol provision device 20, the microphone 25 is configured to output corresponding airflow detection signals to the control circuitry 28. In some embodiments, the microphone 25 is configured to continuously output airflow detection signals, or to output airflow detection signals periodically, such as every 0.01 seconds, every 0.1 seconds, or every 1 second. If the microphone 25 outputs airflow detection signals periodically, then in some implementations the period between the output of subsequent airflow detection signals may be set equal to or less than the average, or a typical length of an user inhalation (e.g. between 2 to 5 seconds) so as to ensure that an inhalation is not missed. In each case, the airflow detection signals change when a flow of air in the aerosol provision device 20 is detected by the microphone 25. For example, the airflow detection signals could be a binary indication of whether a flow of air in the aerosol provision device 20 is detected or not, for example a “1” to indicate a flow of air in the aerosol provision device 20 has been detected and a “0” to indicate that flow of air in the aerosol provision device 20 has not been detected. This is illustrated in FIG. 3B, which is a graph of airflow detection signal output by the microphone 25 against time. Until time point 302, which corresponds to the time point at which the microphone 25 detects a flow of air in the aerosol provision device 20, the airflow detection signal 305A output by the microphone 25 is “0”. This may either represent no airflow detection signal being output, or an airflow detection signal being output with a value equal to “0”. Between time point 301 and 302, which corresponds to the times during which the microphone 25 detects a flow of air in the aerosol provision device 20 (in other words, the noise level exceeds a detection threshold 301), the airflow detection signal 305B output by the microphone 25 is “1”. In other words, the microphone 25 outputs an airflow detection signal to indicate that airflow through the aerosol provision device 20 is detected. After time point 303, the airflow detection signal 305A output by the microphone 25 is “0”, indicating that the microphone 25 no longer detects airflow through the aerosol provision device 20. As set out above, this may either represent no airflow detection signal being output, or an airflow detection signal being output with a value equal to “0”.

Alternatively, the airflow detection signals could correspond to the noise level detected by the microphone 25. If a detection threshold as described above has been implemented, the airflow detection signals could be set to “0” when the noise level detected by the microphone 25 is less than the detection threshold, or the airflow detection signals could include the noise level detected by the microphone 25 along with an indication that the noise level is less than the detection threshold. FIG. 3C illustrates the former example where the airflow detection signals 305A are set to “0” when the noise level detected by the microphone 25 does not exceed the detection threshold 301, and the airflow detection signal 305C corresponds to the noise level detected by the microphone 25 when the noise level detected by the microphone 25 exceeds the detection threshold 301.

In some embodiments, the microphone 25 is configured to only output airflow detection signals when the noise level exceeds the detection threshold. In other worlds, the microphone 25 is configured output airflow detection when the noise level detected by the microphone 25 exceeds the detection threshold, and the microphone 25 is configured to stop outputting airflow detection signals when the noise level does not exceed, or no longer exceeds, the detection threshold. As described above, in this example the portions of the graphs in FIGS. 3A and 3B where the airflow detection signal 305A is zero represent the times when no air flow detection signals are output by the microphone 25.

As described above, the microphone 25 is configured to output the airflow detection signals to the control circuitry 28. In response to receiving the airflow detection signals, the control circuitry 28 is configured to determine a duration of an inhalation by a user of the aerosol provision device 20 based on the airflow detection signals received from the microphone 25. In other words, the control circuitry 28 is configured to determine the elapsed time for an inhalation based on the airflow detection signals received from the microphone 25. As set out above, the microphone 25 may be configured to continuously or periodically output airflow detection signals to the control circuitry 28, and the control circuitry 28 is configured to use the change in these signals described above to determine a duration of an inhalation, for example by starting an inhalation timer when the airflow detection signals change a first time and stop the inhalation timer when the airflow detection signals change a second time. The control circuitry 28 may be configured to start the inhalation timer when the first non-zero airflow detection signal is received, or when the first airflow detection signal indicative of the noise level detected by the microphone 25 exceeding a detection threshold is received, such as at time 302 in FIGS. 3B and 3C respectively. The control circuitry 28 may then be configured to stop the inhalation timer when the next zero value airflow detection signal is received, or when the next airflow detection signal indicative of the noise level detected by the microphone 25 not exceeding the detection threshold is received, such as at time 303 in FIGS. 3B and 3C respectively. Using FIGS. 3A-3C as an example, the duration of inhalation determined by the control circuitry 28 is the elapsed time between the time points 302 and 303.

As set out above, the microphone 25 may be configured to only output airflow detection signals when the flow of air has been detected. In this case, the control circuitry 28 can be configured to determine the duration of the inhalation by activating the inhalation timer when an airflow detection signal is received, and stopping the inhalation timer when airflow detection signals are no longer received.

Alternatively, the duration of an inhalation may be determined based on information contained within the airflow detection signals, such as a time stamp associated with each airflow detection signal. For example, in the case illustrated in FIG. 3B or 3C, the control circuitry 28 is configured to use the timestamp of a first non-zero airflow detection signal (at time 302) received and the timestamp of the next zero value airflow detection signal received (at lime 303) to determine the duration of the inhalation. Alternatively, the control circuitry 28 is configured to use the timestamp of the first airflow detection signal indicative of the noise level detected by the microphone 25 exceeding the detection threshold received, and the timestamp of the next airflow detection signal indicative of the noise level detected by the microphone not exceeding the detection threshold received to determine the duration of the inhalation.

In the example where the airflow detection signals are output periodically by the microphone 25, the control circuitry 28 may be configured to determine the duration of an inhalation by counting the number of consecutive, non-zero airflow detection signals received, or the number of consecutive airflow detection signals indicative of the noise level detected by the microphone 25 exceeding a detection threshold received. The period of output of the airflow detection signals can then be used to determine the duration of the inhalation.

In the example illustrated in FIG. 2C where there are two or more microphones 25A, 25B, each microphone 25 is configured to output airflow detection signals in accordance with the principles described above. The control circuitry 28 is then configured to determine the duration of an inhalation based on the airflow detection signals received from one or more of the microphones 25A, 25B. For example, the control circuitry 28 may be configured to determine the duration of an inhalation in response to receiving airflow detection signals indicating a flow of air has been detected from any one of the microphones 25A, 25B. Alternatively, the control circuitry 28 may be configured to determine the duration of an inhalation in response to receiving airflow detection signals indicating a flow of air has been detected from more than a given percentage of the total number of microphones 25, such as 25%, 50%, 80% or 100%.

In some embodiments, the control circuitry 28 is configured to determine a time between inhalations based on the airflow detection signals. In other words, the control circuitry 28 is configured to determine the elapsed time between an inhalation and the next inhalation. This can be achieved using the same techniques as described above with respect to determining the duration of an inhalation, such as using a timer, information contained within the airflow detection signals or the period of output of the airflow detection signals. For example, the control circuitry 28 may be configured to start an interval timer in response to receiving the first zero value airflow detection signal after a non-zero airflow detection signal. The control circuitry 28 is then configured to stop the interval timer in response to receiving the next non-zero value airflow detection signal.

In the embodiment described above where the microphone 25 is configured to stop outputting airflow detection signals when the noise level does not exceed the detection threshold, the control circuitry 28 can be configured to determine a time between inhalations by activating the interval timer when the microphone 25 stops outputting the airflow detection signals. In other words, the control circuitry 28 is configured to start the interval timer in response to the microphone 25 stopping the output of airflow detection signals following a flow of air in the aerosol provision device 20 having been detected by the microphone 25. The control circuitry 28 can then be configured to stop the interval timer when the next airflow detection signal is outputted by the microphone 25, thereby allowing the control circuitry 28 to determine the time between inhalations.

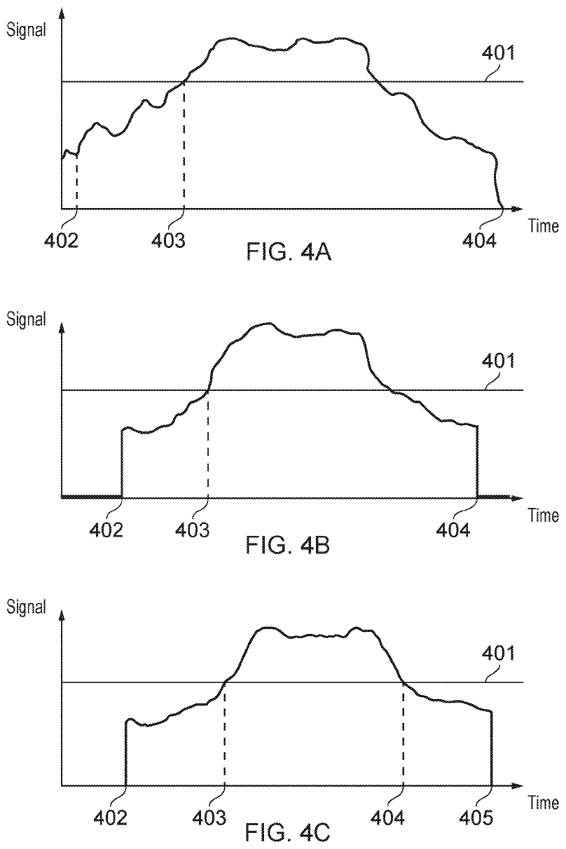

In some embodiments, the control circuitry 28 is configured to detect a first phase of the inhalation and a second phase of the inhalation based on the airflow detection signals. The first phase of inhalation correspond to the portion or phase of the inhalation immediately after the airflow detection signals are received from the microphone, or immediately after when the airflow detection signals change as described above to indicate that an inhalation has been detected. Alternatively, the first phase of the inhalation may begin a predetermined time after the airflow detection signals are received from the microphone 25, or a predetermined time the airflow detection signals change as described above. FIGS. 4A to 4C illustrate graphs of airflow detection signal output by the microphone 25 against time. In the example illustrated in FIG. 4A, the control circuitry 28 starts receiving airflow detection signals from the microphone 25 at time 0. The first phase of the inhalation is then determined to begin a predetermined time after the airflow detection signals are received by the control circuitry 28 from the microphone 25, corresponding to time 402 in FIG. 4A. In FIG. 4B, the first phase of the inhalation is then determined to begin at time 402, which corresponds to the time when the first non-zero airflow detection signal is received from the microphone. In FIG. 4C, first phase of the inhalation is then determined to begin at time 402, which corresponds to the time at which the first airflow detection signal is received from the microphone as described above in relation to FIG. 3.

In some embodiments, the first phase of the inhalation then occurs until the airflow detection signals are above a phase threshold 401. In other words, the first phase of the inhalation is the period from the beginning of the inhalation until the airflow detection signals exceed a phase threshold 401. This is indicated by time 403 in each of FIGS. 4A-C, such that the first phase of inhalation is the period between time 402 and time 403 in each of FIGS. 4A-C. The second phase of the inhalation then occurs after the airflow detection signals are above the phase threshold 401. The second phase of inhalation may continue until the end of the inhalation, for example until the microphone 25 stops outputting airflow detection signals or until the airflow detection signals change as described above to indicate that an inhalation is no longer detected. This illustrated in FIGS. 4A and 4B at time point 404, such that the second phase of the inhalation is the period between time points 403 and 404. Alternatively, the second phase of the inhalation may correspond to the portion of the inhalation where the airflow detection signals exceed the phase threshold 401. In other words, the second phase of the inhalation ends when the airflow detection signals no longer exceed the phase threshold 401. This is illustrated in FIG. 4C, where in time point 404 corresponds to the first time when the airflow detection signal no longer exceeds the phase threshold 401. The second phase of the inhalation is therefore the period between time points 403 and 404.

In some embodiments, there may be more than two phases to the inhalation, for example 5 or 10. In this case, the second phase of the inhalation may end when the airflow detection signals exceed a second phase threshold. A third phase of the inhalation then occurs after the airflow detection signals exceed the second phase threshold. It will be appreciated that the control circuitry may be configured to determine the occurrence of such phases of the inhalation for any number of phases based on the airflow detection signals. In the example illustrated in FIG. 4C, the period after the end of the second phase of the inhalation may correspond to a third phase of the inhalation. The third phase of inhalation may continue until the end of the inhalation, for example until the microphone 25 stops outputting airflow detection signals or until the airflow detection signals change as described above to indicate that an inhalation is no longer detected. This illustrated in FIG. 4C at time point 405, such that the third phase of the inhalation is the period between time points 404 and 405. Alternatively, as the airflow detection signal is below the phase threshold 401 for the period between time points 404 and 405, this period may be determined to be a continuation of the first phase of the inhalation by the control circuitry 28.

In some examples, the control circuitry 28 is also configured to determine the duration of each phase of the inhalation. For example, in the examples illustrated in FIGS. 4A and 4B where there are two phases to the inhalation, the control circuitry 28 is configured to determine the duration of the first phase of the inhalation, between time points 402 and 403, and duration of the second phase of the inhalation, between time points 403 and 404, based on the airflow detection signals. This can be achieved using any of the methods described above with reference the puff duration, such as using one or more timers, the period of the output of the airflow detection signals or information contained in the airflow detection signals, such as a time stamp. The methods used to determine the duration of each phase of the inhalation may be different. For example, a time stamp in the airflow detection signals may be used to determine the duration of the first phase of the inhalation, whilst a timer may be used to determine the duration of the second phase of the inhalation.

In the example illustrated in FIG. 2C where there is more than one microphone 25, the control circuitry 28 may be configured to detect a first phase of the inhalation and a second phase of the inhalation based on the airflow detection signals received from one or more of the microphones. For example, the first phase of the inhalation may correspond to the period when a flow of air is detected at one microphone 25A, whilst the second phase of the inhalation may correspond to the period when a flow of air is detected at more than one microphone 25A, 25B. For example, if there is a microphone located proximate to the inlet 21 and microphone located proximate to the air channel 23, the first phase of the inhalation may correspond to the period when a flow of air is detected at the microphone located proximate to the inlet, and the second phase of the inhalation may correspond to the period when a flow of air is detected at both the microphones.

In some embodiments, the control circuitry 28 is configured to control an operational parameter of the aerosol provision system 10 based on the airflow detection signals. For example, the control circuitry 28 could be configured to activate an indicator light or emit a sound from a speaker on the aerosol provision device 20 or the consumable 30 in response to receiving airflow detection signals in order to indicate to the user that the an inhalation has been detected.

Alternatively, or in addition, the control circuitry 28 may be configured to activate the aerosol generator 36 in response to receiving airflow detection signals. In other words, the control circuitry 28 is configured to signal to the power source 14 or otherwise activate the power source 14 to supply an amount of electrical power to the aerosol generator 36 in response to receiving airflow detection signals. This may prevent the aerosol generator 36 from being inadvertently activated when the user is not inhaling on the aerosol provision system 10. For example, if the aerosol generator 36 is activated in response to an input on a push button, the aerosol generator 36 could be activated inadvertently when the user is fiddling with the aerosol provision system 10, or if the aerosol provision system 10 is dropped or knocked. By activating the aerosol generator 36 in response to receiving airflow detection signals, the aerosol generator 36 is activated when an inhalation is detected.

In some embodiments the control circuitry 28 may be configured to activate or switch on the aerosol generator 36 in response to the airflow detection signals exceeding an activation threshold. The activation threshold may correspond to the same noise level as the detection threshold (in other words the activation threshold and the detection threshold are the same), or the activation threshold may correspond to a higher noise level than the detection threshold. For example, the activation threshold may correspond to the phase threshold as described above. This means that a user is able to puff or inhale on the aerosol provision system 10, but aerosol is only generated when the inhalation has sufficient strength or air speed for the air flow signals to exceed the activation threshold. The activation threshold may depend on the type of consumable 30 that has been attached to the aerosol provision device 20, or may be set and changed by the user.

The control circuitry 28 may also be configured to deactivate or switch off the aerosol generator 36 in response to the airflow detection signals no longer exceeding the activation threshold. In other words, when the airflow detection signals stop exceeding the activation threshold, the control circuitry 28 is configured to signal to the power source 14 or otherwise stop the supply of electrical power to the aerosol generator 36. Alternatively, the control circuitry 28 may be configured to deactivate or switch off the aerosol generator 36 in response to airflow detection signals no longer being output by the microphone 25, or the airflow detection signals indicating that a flow of air is no longer detected in the aerosol provision device 20 by the microphone 25.

The control circuitry 28 may also be configured to control an operational parameter of the aerosol provision system 10 based on the phase of the inhalation as described above. For example the color of the indicator light or the number of indicator lights activated or the pitch and/or duration of a sound emitted from a speaker may change depending on the current phase of inhalation detected by the control circuitry 28. Alternatively or in addition, the amount of electrical power delivered to the aerosol generator 36 may be different for one or more phases of the inhalation. For example, the amount of electrical power delivered to the aerosol generator 36 may be higher for the second phase of inhalation than the first phase of inhalation. Where the activation threshold corresponds to the phase threshold as described above, the amount of electrical power delivered to the aerosol generator 36 during the first phase of inhalation is zero.

Alternatively or in addition, control circuitry 28 may also be configured to control an operational parameter of the aerosol provision system 10 based on the determined duration of the inhalation or one or more phases of the inhalation. For example, a red indicator light may be activated or a sound emitted from a speaker when the inhalation duration exceeds a safety threshold, such as 10, 20 or 30 seconds. Equally, the control circuitry may be configured to stop the delivery of electrical power to the aerosol generator 36 or otherwise deactivate the aerosol generator 36 when the inhalation duration exceeds the safety threshold. An equivalent phase safety threshold may be used in a similar manner for each phase of the inhalation. Alternatively or in additional, the control circuitry 28 may also be configured to reduce the amount of electrical power delivered to the aerosol generator during the duration of the inhalation or the duration of a phase of the inhalation. In other words, at the start of the inhalation or phase of inhalation the electrical power delivered to the aerosol generator 36 is set to a first level or power setting, and as the inhalation or phase of inhalation continues the electrical power or power setting is decreased below the first level. This may occur in a continuous or in a step-wise manner.

In some embodiments, the control circuitry 28 is configured to determine default inhalation behavior for the user based on the airflow detection signals received for a plurality of inhalations. In other words, the control circuitry is configured to detect patterns in the inhalations by the user based on data determined for the inhalations, such as the duration of an inhalation, a time between inhalations, the duration of the first phase and second phase of the inhalation in order to determine, the amount of electrical power delivered to the aerosol generator 36 during the inhalation, a power level or setting for the aerosol generator 36 for the inhalation and the flavorant, type and/or concentration of the aerosol-generating material 38.

The control circuitry 28 may be configured to alter one or more thresholds described above based on the determined default inhalation behavior, such as the detection threshold, the phase threshold, the activation threshold, the safety threshold and the phase safety threshold. The control circuitry 28 may also be configured to alter an operational parameter of the aerosol generation system 10 or aerosol provision device 20 based on the determined default inhalation behavior, such as an amount of electrical power supplied to the aerosol generator 36 by the power source 14, or the color and/or number of light indicators illuminated and/or the volume, pitch and/or duration of a sound emitted on the aerosol provision device 20 for an inhalation.

In some examples, the control circuitry 28 is configured to determine an amount of an ingredient delivered from the aerosol-generating material 38 to the user during the inhalation based on the airflow detection signals. For example, the duration of the inhalation and/or the duration of the first phase of the inhalation and duration of the second phase of the inhalation may be used to determine the amount of the ingredient delivered from the aerosol-generating material 38 to the user during the inhalation. The control circuitry 28 may be configured to use an algorithm or look-up table to determine the amount of the ingredient delivered during the inhalation based on the duration of the inhalation and/or the duration of one or more phases of the inhalation. As described above, the ingredient may be an active substance, such as nicotine, a flavorant, an aerosol-former material or a functional material such as a pH regulator, coloring agent, preservative, binder, filler, stabilizer or antioxidant.

The air velocity or other parameter of the air flow may also be used in the determination of the amount of the ingredient delivered during the inhalation. For example, the airflow detection signals may indicate the magnitude or level of the noise detected by the microphone 25, and the control circuitry 28 is configured to determine the air velocity or mass flow of air in the air channel 23 based on the airflow detection signals. This can then be used in conjunction with the duration of the inhalation or phases of the inhalation to determine the volume of air drawn during the inhalation or other fluid property of the inhalation which can be used to determine the amount of the ingredient delivered during the inhalation. Further, an operational parameter of the aerosol provision system 10, such as the amount of electrical power delivered to the aerosol generator 36 during the inhalation, may also be used to determine the amount of the ingredient delivered from the aerosol-generating material 38 to the user during the inhalation.

In the embodiments described above where the control circuitry 28 is configured to determine one or more phases of the inhalation, the control circuitry 28 may only utilize airflow detection signals corresponding to certain phases of the inhalation to determine the amount of the ingredient delivered during the inhalation. For example, the control circuitry 28 may only consider the airflow detection signals corresponding to phases of the inhalation where the airflow detection signals are above a threshold, such as the phase threshold or activation threshold, in determining the amount of the ingredient delivered during the inhalation. In the case of the phase threshold, this results in only the second phase of the inhalation being used in the determination of the amount of the ingredient delivered during the inhalation, whilst in the case of the activation threshold this results in only the portion of the inhalation corresponding to when the aerosol generator was activated being used to determine the amount of the ingredient delivered during the inhalation. This may be useful in cases where the airflow indication signals indicate that air is flowing through the aerosol provision device 20, but not at a sufficient speed or mass flow to effectively deliver aerosol to the user.

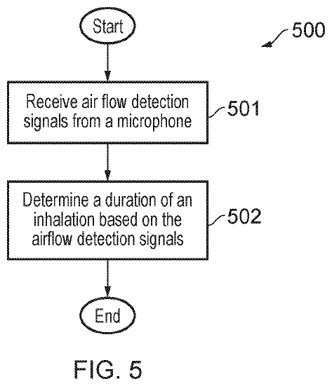

FIG. 5 is a flow chart of a method 500 of determining a duration of an inhalation by a user of an aerosol provision device 20, such as the aerosol provision device 20 described above. The method starts at 501 where airflow detection signals are received from a microphone 25 configured to detect a flow of air in the aerosol provision device 20. At 502 a duration of an inhalation by a user of the aerosol provision device 20 is determined based on the airflow detection signals received from the microphone 25.

The method 500 illustrated in FIG. 5 may be stored as instructions on a computer readable storage medium, such that when the instructions are executed by a processor, the method 500 described above is performed. The computer readable storage medium may be non-transitory.

As described above, the present disclosure relates to (but it not limited to) an aerosol provision device 20 comprising a microphone 25 configured to detect a flow of air in the aerosol provision device 20 and to output corresponding airflow detection signals to control circuitry 28, and control circuitry 28 configured to determine a duration of an inhalation by a user of the aerosol provision device 20 based on the airflow detection signals received from the microphone 25.

Thus, there has been described an aerosol provision device, an aerosol provision system and a method of determining a duration of an inhalation by a user of an aerosol provision device.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the disclosure as defined by the claims or limitations on equivalents to the claims, and that other embodiments may be utilized and modifications may be made without departing from the scope of the claimed disclosure. Various embodiments of the disclosure may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An aerosol provision device comprising:
a microphone configured to detect a flow of air in the aerosol provision device and to output corresponding airflow detection signals to control circuitry; and
control circuitry configured to determine a duration of an inhalation by a user of the aerosol delivery device based on the airflow detection signals received from the microphone.

2. The aerosol provision device of claim 1, wherein the control circuitry is configured to determine a time between inhalations based on the airflow detection signals.

3. The aerosol provision device of claim 1, wherein the microphone is configured to detect the flow of air in the aerosol provision device based on a noise level exceeding a detection threshold.

4. The aerosol provision device of claim 3, wherein the control circuitry is configured to determine the duration of the inhalation by activating an inhalation timer in response to receiving the airflow detection signals from the microphone.

5. The aerosol provision device of claim 3, wherein the microphone is configured to stop outputting airflow detection signals when the noise level does not exceed the detection threshold.

6. The aerosol provision device of claim 5, wherein the control circuitry is configured to determine a time between inhalations by activating an interval timer when the microphone stops outputting the airflow detection signals.

7. The aerosol provision device of claim 1, wherein the control circuitry is configured to control an aerosol generator based on the airflow detection signals.

8. The aerosol provision device of claim 7, wherein the control circuitry is configured to activate the aerosol generator in response to the airflow detection signals exceeding an activation threshold.

9. The aerosol provision device of claim 1, wherein the control circuitry is configured to detect a first phase of the inhalation and a second phase of the inhalation based on the airflow detection signals.

10. The aerosol provision device of claim 9, wherein the control circuitry is configured to determine a duration of the first phase of the inhalation and a duration of the second phase of the inhalation based on the airflow detection signals.

11. The aerosol provision device of claim 9, wherein the first phase of the inhalation occurs until the airflow detection signals are above a phase threshold.

12. The aerosol provision device of any one of claim 11, wherein the second phase of the inhalation occurs after the airflow detection signals are above the phase threshold.

13. The aerosol provision device of any one of claim 1, wherein the control circuitry is configured to determine an amount of an ingredient delivered from an aerosol-generating material to the user during the inhalation based on the airflow detection signals.

14. The aerosol provision device of claim 9, wherein the control circuitry is configured to determine an amount of an ingredient delivered from an aerosol-generating material to the user during the inhalation based on a duration of the first phase of the inhalation and a duration of the second phase of the inhalation.

15. The aerosol provision device of claim 13, wherein the ingredient is nicotine.

16. The aerosol provision device of claim 1, wherein the control circuitry is configured to determine default inhalation behavior for the user based on the airflow detection signals received for a plurality of inhalations.

17. The aerosol provision device of claim 16, wherein the control circuitry is configured to activate the aerosol generator in response to the airflow detection signals exceeding an activation threshold, and the control circuitry is configured to alter the activation threshold based on the determined default inhalation behavior.

18. The aerosol provision device of claim, wherein the control circuitry is configured to detect a first phase of the inhalation and a second phase of the inhalation based on the airflow detection signals, wherein the first phase of the inhalation occurs until the airflow detection signals are above a phase threshold, and the control circuitry is configured to alter the phase threshold based on the determined default inhalation behavior.

19. An aerosol provision system comprising the aerosol provision device of claim 1.

20. An aerosol provision system comprising:
a microphone configured to detect a flow of air in the aerosol provision device and to output corresponding airflow detection signals to control circuitry; and
control circuitry configured to determine a duration of an inhalation by a user of the aerosol provision device based on the airflow detection signals received from the microphone.

21. A method of determining a duration of an inhalation by a user of an aerosol provision device comprising:
receiving airflow detection signals from a microphone configured to detect a flow of air in the aerosol provision device; and
determining a duration of an inhalation by a user of the aerosol provision device based on the airflow detection signals received from the microphone.

22. A non-transitory computer readable storage medium comprising instructions which, when executed by a processor, cause the processor to perform a method comprising:
receiving airflow detection signals from a microphone configured to detect a flow of air in the aerosol provision device; and
determining a duration of an inhalation by a user of the aerosol provision device based on the airflow detection signals received from the microphone.
